(19) United States
(12) Patent Application Publication
(10) Pub. No.: US 2008/0300484 A1
(43) Pub. Date: Dec. 4, 2008

(54) Delay insensitive SVD algorithm for perfusion analysis

(76) Inventors: Jing Wang, Eden Prairie, MN (US); Osama Masoud, Woodbury, MN (US); Brian Frake, Delano, MN (US)

Correspondence Address: SCHWEGMAN, LUNDBERG & WOESSNER, P.A., P.O. BOX 2938, MINNEAPOLIS, MN, 55402, US

(21) Appl. No.: 12/082,695

(22) Filed: Apr. 11, 2008

Related U.S. Application Data
(60) Provisional application No. 60/923,402, filed on Apr. 13, 2007.

(51) Int. Cl. A61B 5/026; A61B 6/00

(52) U.S. Cl. 600/431; 382/128

(57) ABSTRACT

This document discusses, among other things, a computerized system comprising a perfusion analyzer circuit and a memory circuit to store a time sequence of volumetric image data. The image data is obtained in a time relation to arrival of a contrast agent at an anatomical region associated with the image. The perfusion analyzer circuit is configured to access the stored image data and receive a representation of an artery and a representation of tissue near the artery, shift the representation of the artery in time such that a time of arrival of the contrast at the artery precedes arrival of the contrast at the representation of tissue, automatically compute a perfusion parameter using the shifted representation of the artery, and provide the perfusion parameter as an output to a user or to an automated process.

### RELATED APPLICATION

This document claims priority to Provisional Application Ser. No. 60/923,402 (Attorney Docket No. 543.037PRV), titled “Delay Insensitive SVD Algorithm for Perfusion Analysis,” which was filed Apr. 13, 2007, and which is incorporated by reference herein.

### BACKGROUND

The standard singular value decomposition (sSVD) method is one of the major deconvolution techniques useful for brain perfusion analysis of datasets acquired from computed tomography (CT) and magnetic resonance (MR). The method can calculate a contrast residue function, such as for each volume of interest (VOI), using a selected artery curve and the tissue curve at the VOI. The computed residue function can then be used to compute the relative cerebral blood flow (rCBF) and the mean transit time (MTT). However, the standard SVD (sSVD) technique cannot handle cases where there are delays in the times of arrival of the contrast agent to different areas of the brain, in particular when the artery curve is selected from a region to which contrast agent is delayed. The present inventors have recognized a need for improved perfusion analysis of such data sets.

### OVERVIEW

This document relates generally to systems, devices, and methods for image processing. In example 1, a computerized system includes a memory circuit, configured to store a time sequence of volumetric image data, and a perfusion analyzer circuit in communication with the memory circuit. The stored image data is obtained in a time relation to arrival of a contrast agent at an anatomical region associated with the image. The perfusion analyzer circuit is configured to access the stored image data and receive a representation of at least a portion of an artery included in the image data and a representation of tissue near the artery in the image data, shift the representation of the artery in time such that a time of arrival of the contrast at the artery precedes arrival of the contrast at the representation of tissue, automatically compute a perfusion parameter using the shifted representation of the artery, and provide the perfusion parameter as an output to a user or to an automated process.

In example 2, the perfusion analyzer circuit of example 1 optionally comprises a mean transit time (MTT) computation circuit that is configured to determine a MTT of the contrast using the computed perfusion parameter.

In example 3, the MTT computation circuit of example 2 optionally comprises a filter circuit to inhibit oscillations in determined MTT values. In example 4, the filter circuit of example 3 is optionally configured to extend a length of the representation of the artery by zero-padding to inhibit oscillations in determined MTT values.

In example 5, the perfusion analyzer circuit of examples 1-4 optionally comprises a MTT computation circuit that is configured to precondition a system of linear equations to model a representation of infinite zero-padding of the shifted artery to inhibit oscillations in determined MTT values, solve the preconditioned system of linear equations to compute the perfusion parameter, and determine values of MTT of the contrast using the perfusion parameter computed by solving the preconditioned system of linear equations.

In example 6, the representation of infinite zero-padding of example 5 optionally includes a Toeplitz matrix of values of the shifted artery. In example 7, the perfusion analyzer circuit of examples 5-6 is optionally configured to compute the perfusion parameter by solving a preconditioned system of linear equations using standard singular value decomposition.

In example 8, the perfusion analyzer circuit of examples 1-7 is optionally configured to shift the representation of the artery such that the time of arrival of contrast at the artery curve is at a time equal to zero (t=0) for performing deconvolution.

In example 9, the perfusion analyzer circuit of examples 1-8 is optionally configured to compute a contrast residue in a region of a stored image voxel as a function of time. In example 10, the perfusion analyzer circuit of examples 1-9 is optionally configured to automatically compute relative cerebral regional blood flow in a region of a stored image voxel using the perfusion parameter.

In example 11, a computerized method includes accessing a stored time sequence of volumetric image data, wherein the stored image data is obtained in a time relation to arrival of a contrast agent at a region of a subject, receiving a representation of at least a portion of an artery in the stored image data and a representation of tissue near the artery in the image data, shifting the representation of the artery in time such that a time of arrival of the contrast at the artery precedes arrival of the contrast at the representation of tissue, automatically computing a perfusion parameter using the shifted representation of the artery, and providing the perfusion parameter as an output to a user or to an automated process.

In example 12, the method of example 11 optionally includes determining a MTT of the contrast using the computed perfusion parameter. In example 13, the shifting of the representation of the artery of example 12 optionally includes adding zeros to an end of the representation of the artery to extend the artery to inhibit oscillations in determined MTT values.

In example 14, the method of examples 12-13 optionally includes preconditioning a system of linear equations to include a representation to model infinite zero-padding of the shifted artery to inhibit oscillations in determined MTT values, solving the preconditioned system of linear equations for computing the perfusion parameter, and determining MTT of the contrast using the computed perfusion parameter.

In example 15, the preconditioning the system of linear equations of example 14 optionally includes using a representation of infinite zero-padding that includes a Toeplitz matrix of values of the shifted artery.

In example 16, the solving the preconditioned system of linear equations of examples 14-15 optionally includes solving the system using standard singular value decomposition to compute the perfusion parameter.

In example 17, the shifting the representation of the artery of examples 11-16 optionally includes shifting such that the time of arrival of contrast at the artery curve is at a time equal to zero (t=0) for performing deconvolution.

In example 18, the computing the perfusion parameter of examples 11-17 optionally includes computing contrast residue in a region of a stored image voxel as a function of time. In example 19, the method of example 11-18 optionally includes computing relative cerebral regional blood flow in a region of a stored image voxel using the perfusion parameter.

In example 20, a computer-readable medium includes instructions configured to perform a process comprising accessing a stored time sequence of image data, wherein the stored image data is obtained in a time relation to arrival of a contrast agent at a region of a subject, receiving a representation of at least a portion of an artery in the stored image data, shifting the representation of the artery curve in time such that a time of arrival of contrast at the artery precedes arrival of contrast at the representation of tissue, automatically computing a perfusion parameter using the shifted representation of the artery, and providing the perfusion parameter as an output to a user or to an automated process.

In example 21, the computer-readable medium of example 20 optionally includes instructions configured for computing relative cerebral regional blood flow in a region near a stored image voxel using the perfusion parameter.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is a flow diagram of a computerized method of determining a perfusion parameter without adding instability to the results.

FIG. 2 is a block diagram of portions of an example of a computerized system to perform perfusion analysis on image data.

FIG. 3 is a graph showing computed MTT values when different shifts are used.

FIG. 4 shows an artery curve, a shifted artery curve, and a tissue curve.

FIG. 5 shows MTT values that oscillate when smaller number of zeros is used in padding curves.

FIG. 6 shows the sensitivity of the dSVDs algorithm with respect to the zero padding.

FIG. 7 shows results in determining cerebral blood flow (CBF) using the sSVD technique.

FIG. 8 shows results for CBF obtained using the dSVDs technique.

### DETAILED DESCRIPTION

Singular value decomposition (SVD) technique is useful for Computed Tomography (CT) and Magnetic Resonance (MR) brain perfusion analysis. However, if there are delays (e.g., difference of the time of arrivals of the contrast agents) between the artery and tissue curves, the computed mean transit time (MTT) and regional cerebral blood flow (rCBF) might not be correct when the standard SVD (sSVD) algorithm is used. For example, when the delay between the artery curve and a tissue curve is negative, e.g., if the contrast arrives at the tissue curve before it arrives at the selected artery curve, the sSVD algorithm tends to under-estimate the MTT value, which in turn, results in an over-estimation of the CBF. However, the sSVD algorithm can work fine when the delays between the artery curve and the tissues curves are positive.

Several modifications of the sSVD techniques can remedy the problem. One example uses a reformulated SVD algorithm (rSVD) in which the tissue curves are offset to eliminate the delay effects, however the offset used is fixed and can either be too large, which might result in over-estimation of the MTT values, or too small, which might not be enough to completely eliminate the delay effect. In addition, the method may be potentially unstable in the sense that the computed MTT values depend on the amount of offset.

Another method uses circulant deconvolution in which the artery curve and the tissue curves can be extended such as by padding zeros and the SVD technique can be applied on a block-circulant matrix. The bSVD algorithm can eliminate the delay effects of the tissue curves with the expense of additional computation time.

When the SVD technique is used for the deconvolution, singular values that are less than a predefined threshold (P_{SVD}) can be ignored, such as to minimize the oscillation of the computed residue function. For the sSVD, the rSVD and the bSVD algorithms, the threshold P_{SVD} is usually set as a fixed percentage (usually 5%-20%) of the maximum singular value of the convolution matrix. In general, larger P_{SVD} will result in a smoother residue function. However it tends to overestimate the MTT values; while smaller P_{SVD} will generally increase the oscillation of the residue function. To balance the problem, oscillation minimization technique (oSVD) using adaptive thresholds can be used. In this approach, an adaptive threshold can be chosen for each individual tissue curve based on the oscillation of the residue function, an optimal threshold P_{SVD} can be chosen such that the total oscillation of the residue function is less than a given threshold. The algorithm behavior is slightly better than the bSVD algorithm with the cost of additional computation time.

A better approach to deal with delay is to shift the artery curve such as to completely eliminate the possible delay effect. In some examples, the technique includes shifting the artery curve so that the time of arrival of the contrast to the shifted artery curve occurs before that of all the tissue curves.

In certain examples, a pre-conditioning technique can be applied, such as to stabilize the deconvolution calculation. The pre-conditioning technique can be conceptualized as padding infinitely many zeros at the end of the curves with minimal overhead on computation time. Demonstrated results using real datasets verify that the technique correctly handles delays, and eliminates the instability in computed results that is introduced by the shifting of the artery curve.

### Theory

Given an artery input function Cₐ(t) and the transfer function h(t) that represents the distribution of the contrast in the system over time, the mean transit time (MTT) for the tracer particles can be expressed as

MTT=∫₀^{∞}th(t)dt. (2.1)

For a particular pixel x, the regional cerebral blood volume (rCBV), which measures the amount of intra-vascular tracer at the pixel can be determined by

$$rCBV = \frac{{\int }_{0}^{∞} {C}_{x} ( t ) d t}{{\int }_{0}^{∞} {C}_{v} ( t ) d t} , \qquad ( 2.2 )$$

where Cₓ(t) is the contrast concentration curve at the tissue and Cᵥ(t) is the contrast concentration curve at a venous pixel.
Once rCBV and MTT are computed, the regional cerebral blood flow (rCBF) can be calculated as

$$rCVF = \frac{rCBV}{MTT} . \qquad ( 2.3 )$$

The residue function, which measures the fraction of injected tracer remaining in the system, can be written as

R(t)=1−∫h(τ)dτ. (2.4)

Using the residue function $R(t)$, MTT can be calculated as

MTT=1−∫₀^{∞}R(t)dt. (2.5)

The equation (2.5) can be derived from the equation (2.1) by using the fact that

R′(t)=−h(t). (2.6)

### SVD Based Deconvolution Algorithm

In SVD based deconvolution algorithms, the concentration of tracer within a tissue voxel (x) as a function of time (t) can be modeled as

$$\begin{matrix}{C}_{x} ( t ) = {F}_{x} {C}_{a} ( t ) ⊗ {R}_{x} ( t ) \\ = {F}_{x} {\int }_{0}^{t} {C}_{a} ( τ ) {R}_{x} ( t - τ ) d τ ,\end{matrix} \qquad ( 3.1 )$$

where Fₓ is the tissue blood flow at x and Rₓ(t) is the residue function.

### Standard SVD Deconvolution Algorithm

Assume the arterial and cerebral concentrations are measured at a set of equally spaced time points t₀t₁, . . . , t_{N}, then the equation (3.1) can be discretized as

$$\begin{matrix}{C}_{x} ( {t}_{i} ) = {F}_{x} {\int }_{0}^{{t}_{i}} {C}_{a} ( τ ) {R}_{x} ( {t}_{i} - τ ) d τ \\ = Δ {tF}_{x} {\sum }_{k = 0}^{i} {C}_{a} ( {t}_{k} ) {R}_{x} ( {t}_{i} - {t}_{k} ) ,\end{matrix} \qquad ( 3.2 )$$

for i=0, 1, . . . , N.
Written in the matrix form, we have

Cₓ=ΔtFₓMₛRₓ, (3.3)

where

$$\begin{matrix}{M}_{s} = [ \begin{matrix}{C}_{a} ( {t}_{0} ) & 0 & … & 0 \\ {C}_{a} ( {t}_{1} ) & {C}_{a} ( {t}_{0} ) & … & 0 \\ ⋮ & ⋮ & ⋮ & ⋮ \\ {C}_{a} ( {t}_{N} ) & {C}_{a} ( {t}_{N - 1} ) & … & {C}_{a} ( {t}_{0} )\end{matrix} ] , & ( 3.4 ) \\ {C}_{x} = [ \begin{matrix}{C}_{x} ( {t}_{0} ) \\ {C}_{x} ( {t}_{1} ) \\ ⋮ \\ {C}_{x} ( {t}_{N} )\end{matrix} ] , and {R}_{x} = [ \begin{matrix}R ( {t}_{0} ) \\ R ( {t}_{1} ) \\ ⋮ \\ R ( {t}_{N} )\end{matrix} ] . & ( 3.5 )\end{matrix}$$

Note that the matrix Mₛ is a Toeplitz matrix (A N×N matrix M is called a Toeplitz matrix if it has constant diagonals, i.e. if M_{i,j}=M_{i-1,j-1} for all 0<i, j<N).

Using standard SVD technique, the matrix Mₛ can be decomposed as

Mₛ=USV^{T}, (3.6)

where U and V are orthonormal matrices and S is a diagonal matrix with the diagonal elements being the singular values of the matrix Mₛ. The inverse matrix Mₛ⁻¹ can be calculated as

Mₛ⁻¹≈V S̅⁻¹U^{T}, (3.7)

where S̅⁻¹ is a diagonal matrix that approximates the inverse matrix of S, and that can be written as

$${\overline{S}}_{ii}^{-- 1} = \{ \begin{matrix}1 / {S}_{ii} & if {S}_{ii} > {P}_{SVD} \\ 0 & otherwise\end{matrix} \\ i = 0 , … , N . \qquad ( 3.8 )$$

Here P_{SVD} is the cut-off threshold, which is usually set to be a percentage of the maximum singular value of Mₛ. Then we have

\tilde{R}ₓ=ΔtFₓRₓ=Mₛ⁻¹Cₓ≈V S̅⁻¹U^{T}Cₓ. (3.9)

From which, we can calculate the MTT as

$$MTT = \frac{Δ t {\sum }_{i = 0}^{N} \tilde{R} ( {t}_{i} )}{max \tilde{R} ( {t}_{i} )} . \qquad ( 3.10 )$$

It should be noted that the MTT values computed using this technique will highly depend on the choice of the cut-off threshold P_{SVD}. Larger P_{SVD} will result in smoother residue curve, with the sacrifice of less accurate (usually higher) MTT values. Smaller P_{SVD} will give more accurate MTT values, but the computed residue function will be less smooth. Usually, P_{SVD} is set to be in the range of 5%-20% of the maximum singular value of Mₛ.

### Block-Circulant SVD Deconvolution Algorithms

In the block-circulant SVD deconvolution (bSVD) algorithm, the artery curve and all of the tissue curves are extended to a length (L) that is at least twice of the length of the original curves by padding zeros at the end of the curves. Using circular deconvolution, the deconvolution matrix M_{b} (when the curves are extended to be twice of the original length) can be written as

$${M}_{b} = [ \begin{matrix}{M}_{s} & B \\ B & {M}_{s}\end{matrix} ] , \qquad ( 3.11 )$$

Where Mₛ is defined in (3.4) and

$$B = [ \begin{matrix}0 & {C}_{a} ( {t}_{N} ) & {C}_{a} ( {t}_{(} N - 1 ) ) & … & {C}_{a} ( {t}_{1} ) \\ 0 & 0 & {C}_{a} ( {t}_{N} ) & … & {C}_{a} ( {t}_{2} ) \\ ⋮ & ⋮ & ⋮ & ⋮ & ⋮ \\ 0 & 0 & 0 & … & 0\end{matrix} ] . \qquad ( 3.12 )$$

M_{b} is a block-circulant Toeplitz matrix since both the matrix Mₛ and the matrix B are Toeplitz matrices. Again the SVD algorithm is used to solve the system and the formula (3.10) can be used to calculate the mean transit time.

In the bSVD algorithm, similar P_{SVD} cut-off threshold can be used for eliminating the oscillation introduced otherwise by small singular values. However, as we mentioned earlier, smaller P_{SVD} value usually gives more accurate estimate of MTT, thus the cut-off threshold P_{SVD} should generally be chosen as small as possible without sacrificing too much on the smoothness of the residue functions. To this purpose, an oscillation minimization bSVD (oSVD) algorithm can be used, in which an adaptive singular value threshold is chosen for each individual pixel depending on the smoothness of the residue function. For example, the smallest P_{SVD} can be chosen, such that the normalized sum of the approximated curvature of the residue function

$$O = \frac{1}{( N - 1 ) {R}_{max}} [ {\sum }_{i = 1}^{N - 1} \vert {R}_{{t}_{i + 1}} - 2 {R}_{{t}_{i}} + {R}_{ti - 1} \vert ] \qquad ( 3.13 )$$

is less than a given threshold. Here Rₘₐₓ is the maximum value of the residue function R(tᵢ), i=0, 1, . . . N, and Rₜ_{ᵢ}=R(tᵢ). The oSVD algorithm usually gives better MTT estimation with the expense of more computation time.
Delay Insensitive SVD Algorithm Using Shifted Artery (or dSVDs)

The standard SVD algorithm can work fine when the delay of a tissue curve with respect to the selected artery curve is positive, in other words, when the tissue curve is “behind” the artery curve. However when the delay is negative, e.g., when the contrast arrives earlier to a tissue curve than to the selected artery curve, the computed MTT value tends to be much lower than its actual value. Negative delay happens when the artery is selected from a vessel with abnormal flow. For instance, when the left carotid artery is blocked due to stenosis, the vessel from the left side of the brain will have abnormal flow since the blood supply will be from the right carotid artery, in which case, the contrast will arrive earlier to the tissues on the right side of the brain than these on the left side of the brain. An approach to fix the problem shifts the tissue curves so that the delays between the tissue curves and the artery curve are non-negative.

One question in using the approach is that how much the curves should be shifted. If the shift is too small, some tissue curves could still be “before” the artery curve, if the shift is too large, the computed MTT values will be affected. Thus, the algorithm is unstable with respect to the shift. Instead of shifting the tissue curves to eliminate the negative delays, a better approach is to shift the artery curve.

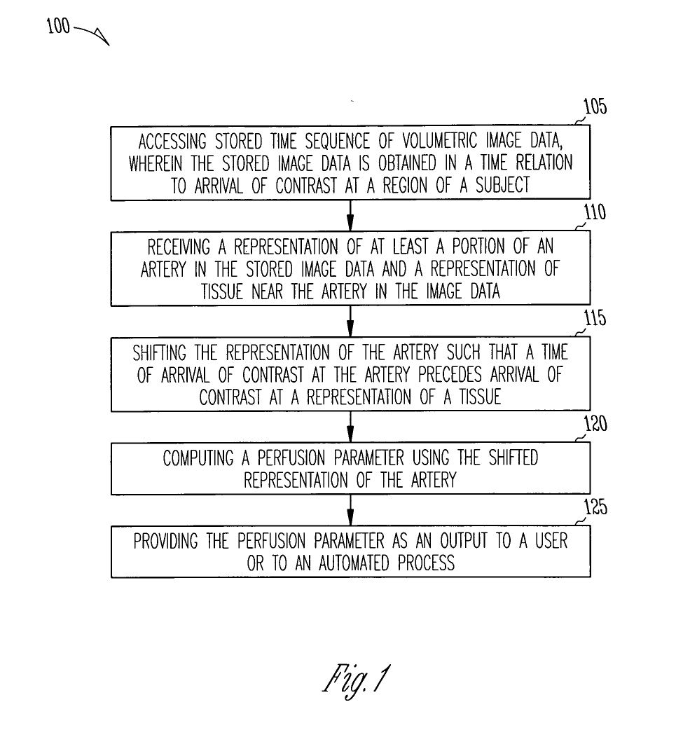

FIG. 1 is a flow diagram of a computerized method 100 of determining a perfusion parameter without adding instability to the results. At block 105, a stored time sequence of volumetric image data is accessed. The stored image data may be accessed from a memory by a processor. The stored image data is obtained in a time relation to arrival of contrast agent at a region of a subject. In some examples, the image data is acquired using computed tomography (CT). In some examples, the image data is acquired using magnetic resonance (MR). In some examples, the image data is used to reconstruct a 3D image of the subject. This 3D image or volume consists of volume elements, or voxels. The image data is acquired in a time relationship to application of a contrast agent to the volume in the image. In some examples, the image includes a cerebral image of the subject.

At block 110, a representation of at least a portion of an artery (e.g., an artery curve) in the stored image data and at least a portion of tissue (e.g., a tissue curve) near the artery is received, such as by a processor for example. At block 115, the representation of the artery is shifted in time such that a time of arrival of contrast at the artery curve precedes arrival of the contrast at the representation of the tissue.

In some examples, an artery curve can be shifted such that the time of arrival of contrast is at t=0. Assume the original artery curve is Cₐ(t), 0≦t≦T and the time of arrival of contrast to the artery curve is to, the new artery curve can be written as

$${\overline{C}}_{a} ( t ) = \{ \begin{matrix}{C}_{a} ( t + {t}_{0} ) & t \leq T - {t}_{0} \\ 0 & otherwise .\end{matrix} \qquad ( 4.1 )$$

At block 120, a perfusion parameter is automatically computed using the shifted representation of the artery, and at block 125 the perfusion parameter is provided as an output to a user or to an automated process. Examples of the perfusion parameter include, among other things, a parameter related to mean transit time (MTT) of the contrast, a parameter related to contrast (sometimes called tracer) residue in a region near an image voxel, and a parameter related to blood flow.

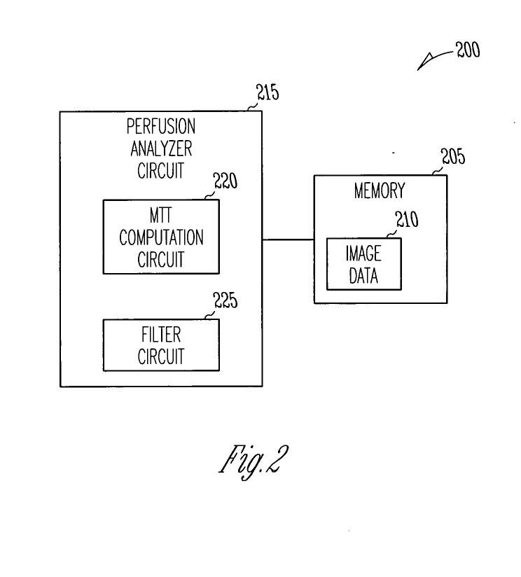

FIG. 2 is a block diagram of portions of an example of a computerized system 200 to perform perfusion analysis on image data. The system 200 includes a memory 205 to store a time sequence of volumetric image data 210. The image data 210 is obtained in a time relation to arrival of a contrast agent at an anatomical region associated with the image. For example, the image data 210 may be acquired by CT or MR in a time relationship to a contrasting agent being injected into the subject's brain.

The system 200 also includes a perfusion analyzer circuit 215 in communication with the memory 205. The perfusion analyzer circuit 215 may include a processor, such as a microprocessor for example. The processor performs or executes a function or functions. Such functions correspond to circuits or other type of modules, which are software, hardware, firmware or any combination thereof. Multiple functions may be performed in one or more of the circuits. The processor may operate on a computer system, such as a personal computer, workstation, server, or other computer system. The perfusion analyzer circuit 215 may be in communication with the memory 205 by exchange of electrical signals for example.

The perfusion analyzer circuit 215 accesses the stored image data 210 and receives a representation of at least a portion of an artery (e.g., an artery curve) included in the image data and a representation of tissue (e.g., a tissue curve) near the artery in the image data. The perfusion analyzer circuit 215 shifts the representation of the artery in time such that a time of arrival of the contrast at the artery precedes arrival of the contrast at the representation of the tissue included in the image data. The perfusion analyzer circuit 215 automatically computes a perfusion parameter using the shifted representation of the artery curve, and provides the perfusion parameter as an output to a user or to an automated process.

In some examples, the perfusion analyzer circuit includes a mean transit time (MTT) computation circuit 220 to determine a MTT of the contrast agent using the computed perfusion parameter.

As the shifted representation of the image data will show the contrast agent arriving at the tissue after t>0, the delays of the tissue curves with respect to the new artery curve will be positive, which is fine for the standard SVD algorithm.

Shifting the artery replaces shifting the tissue curves, and theoretically, the MTT values should not change when the artery curve is shifted. However, due mostly to the cut-off threshold used to remove the small singular values, the computed MTT values will vary when different shifts are used.

FIG. 3 shows the computed MTT values when different shifts (from 0 second to 6 seconds) are used. Notice that when no shift is applied, the computed MTT value is much smaller than those with positive shifts, this is caused exactly by the negative delay. For 1-6 seconds shifts, the MTT values are relatively similar though they still vary from 3.9 seconds to about 5 seconds. This illustrates the instability of the algorithm with respect to the shift. Additional techniques are applied to stabilize the algorithm results.

In some examples, the MTT circuit 220 includes a filter circuit 225 to inhibit the oscillations in determined MTT values introduced by the shifting. The filter circuit 225 extends a length of the representation of the artery and the tissue by zero-padding to inhibit the oscillations and stabilize the determined MTT values. (In the bSVD algorithm, the number of zeros in the padding is at least the length of the original artery or tissue curve (e.g., if the original curve has m temporal point, the new curve should have at least 2 m points). This implies that all of the contrast will be out of the tissues after certain time, which is physiologically realistic. A real dataset may illustrate the effect of the zero padding.

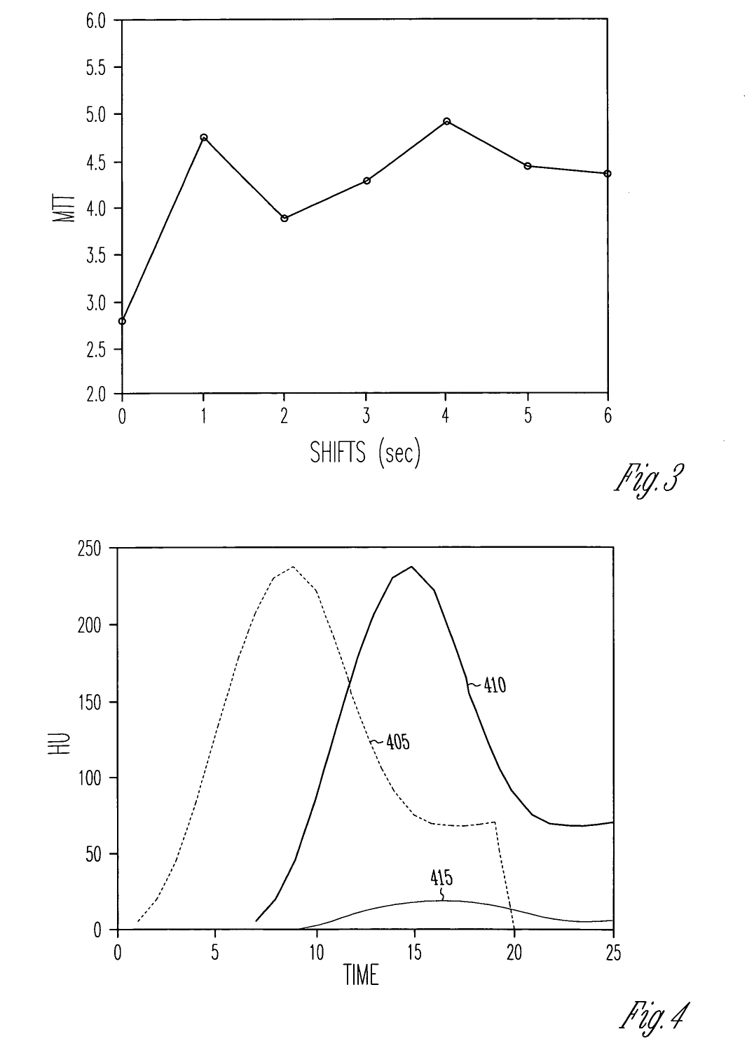

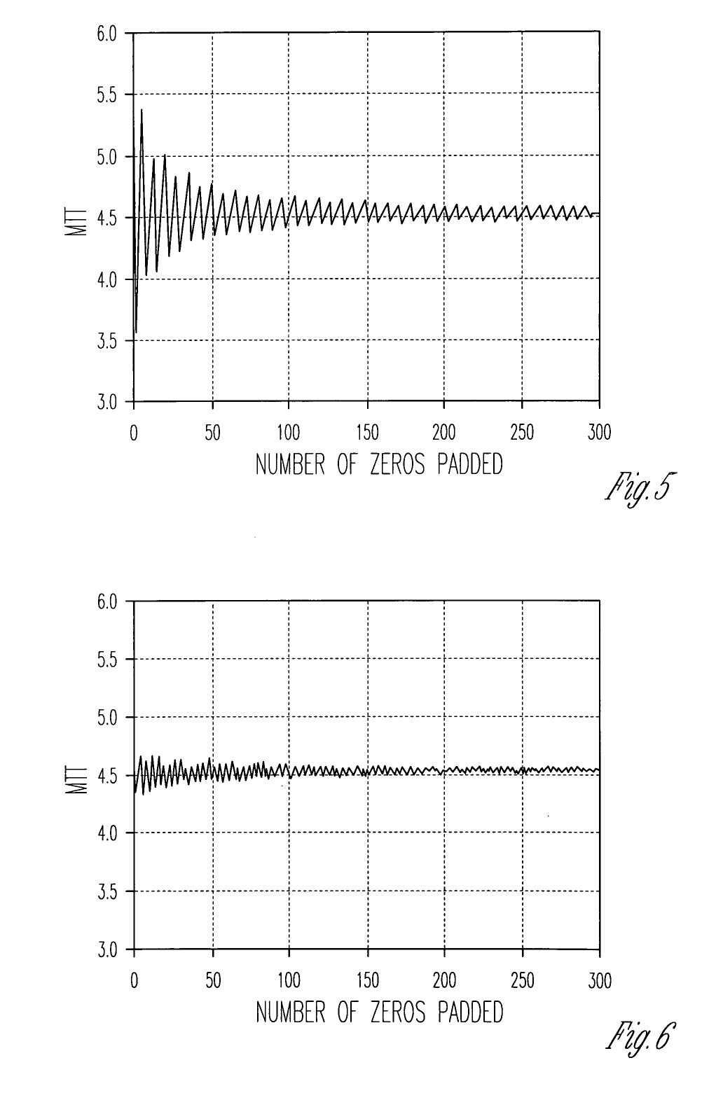

FIG. 4 shows the original artery curve 405, shifted artery curve 410, and one tissue curve 415. FIG. 5 shows the computed MTT values (using sSVD with the shifted artery) with respect to the number of padded zeros.

FIG. 5 shows that the MTT values oscillate dramatically when smaller number of zeros is padded. However the oscillation is always around a fixed value, and when more and more zeros are used, the oscillation gradually decreases. So by padding large number (ideally infinitely many) of zeros, the oscillation can be minimized. One difficulty is determining how many zeros should be used in the padding of the artery curve. When small numbers of zeros are used, the results produced by the algorithm will not be stable enough, as shown for MTT in FIG. 5. When a large number of zeros is added to minimize the oscillation, the artery curve will be longer, the artery matrix will be larger and the computation will be more expensive.

In some example, the MTT computation circuit 220 uses a pre-conditioning technique to stabilize the algorithm without padding any zeros. The MTT computation circuit 220 computes the perfusion parameter by solving a system of linear equations. The MTT computation circuit 220 preconditions the system of linear equations to model a representation of infinite zero-padding of the shifted artery curve to inhibit oscillations in determined MTT values.

When the artery is shifted according to (4.1), the first element of the artery curve will be non-zero, thus the artery matrix (For simplicity, we still denote it as Mₛ) is non-singular. So solving the linear system

Cₓ=MₛRₓ (4.2)

will be a viable alternative to solving the system

Mₛ^{T}Cₓ=Mₛ^{T}MₛRₓ=Mₛ²Rₓ. (4.3)

Note that the singular values of Mₛ² will be exactly the squares of the singular values of the matrix Mₛ since if

Mₛ=USV^{T}, (4.4)

then

Mₛ²=Mₛ^{T}*Mₛ=VSU^{T}USV^{T}=VS²V^{T}. (4.5)

Therefore, if the cut-off threshold for Mₛ is P_{SVD}, the cut-off threshold for the new matrix Mₛ² should be P_{SVD}².

Using the SVD technique, the equation (4.3) will give the same solution as the equation (4.2) will produce no matter what P_{SVD} is chosen. So simply pre-conditioning the equation (4.2) with Mₛ^{T} will not take care of the stability issue.

Note that the matrix Mₛ² is symmetric, but it is not a Toeplitz matrix. Since the solutions of the (4.2) and (4.3) are the same, the system (4.3) can also be stabilized using the zero-padding technique. One interesting fact is that if the artery curve C̅ₐ(t) is padded using infinite number of zeros, the resulting matrix (Mₛ^{∞})² will be a Toeplitz matrix. To take advantage of the stabilizing effect of infinite zero padding without actually padding any zeros, we modify the matrix Mₛ² such that it becomes a Toeplitz matrix. In other words, we replace the Mₛ² with the (N+1)×(N+1) upper-left sub-matrix (we denote it as \hat{M}ₛ² of (Mₛ^{∞})². Then the linear system becomes

Mₛ^{T}Cₓ=\hat{M}ₛ²Rₓ. (4.6)

Again, we use SVD technique to solve the system for Rₓ, and MTT is calculated using the formula (2.5). In some examples, the MTT computation circuit 220 uses a combination of zero padding and pre-conditioning. The technique may be referred to as delay-insensitive SVD technique using shifted artery, or dSVDs.

An example of the dSVDs technique is summarized below.

1. Artery Shifting
The artery is shifted so that the time of arrival of contrast is at t=0. Zeros are added when the artery curve is shifted.

2. Linear System Formulation
Instead of solving the standard linear system (4.2), we solved the pre-conditioned system (4.6). The matrix \hat{M}ₛ² is formulated below.
Let the column vector of the matrix Mₛ be denoted as vᵢ, i=0, . . . , n.
We compute the dot product of v₀ and vᵢ for i=0, . . . , n as d(i)=v₀^{T}v, d(i)=0, . . . , n.
The matrix \hat{M}ₛ² is defined as

\hat{M}ₛ²(i,j)=d)|i−j|) (4.7)

Matrix \hat{M}ₛ²² is a symmetric and positive definite Toeplitz matrix.

3. Deconvolution

The linear system (4.6) can be solved using the standard SVD technique. We compute the SVD decomposition \hat{M}ₛ²=ÛŜ\hat{V}^{T}. As usual, S⁻¹ can be calculated using (3.8). Note that if the cut-off percentage is 5% for the original matrix Mₛ, the cut-off percentage for the matrix \hat{M}ₛ² should be (5%)²=0.25%. Rₓ can be calculated as Rₓ=\hat{V}Ŝ⁻¹Û^{T}Mₛ^{T}Cₓ.

FIG. 6 shows the sensitivity of the dSVDs algorithm with respect to the zero padding, using the curves in FIG. 4.

Even though the computed MTT values may still oscillate, the oscillation is much smaller compare with the oscillation seen in FIG. 3. In addition, the oscillation is around the same value as before, this confirms that the pre-conditioning technique stabilizes the algorithm without changing the solution of the original equation. Table 1 shows statistics of the MTTs computed using the two algorithms when up to 300 zeros are padded to the artery and tissue curves.

TABLE 1

| MTT | min | max | mean | std | max-min |
|---|---|---|---|---|---|
| sSVDs | 3.46 | 5.87 | 4.53 | 0.17 | 2.41 |
| dSVDs | 4.32 | 4.67 | 4.52 | 0.05 | 0.35 |

It can be seen from the table that while the mean values are very similar as expected, the standard deviations (std) are different dramatically (the std for the sSVDs algorithm is more than 3 times larger than that for the dSVDs algorithm). In addition, the variation of MTT is much smaller for the dSVDs algorithm (0.35) compared to the variation for the sSVDs algorithm (2.41). This illustrates that the dSVDs algorithm is much more stable than the sSVDs algorithm.

### Results

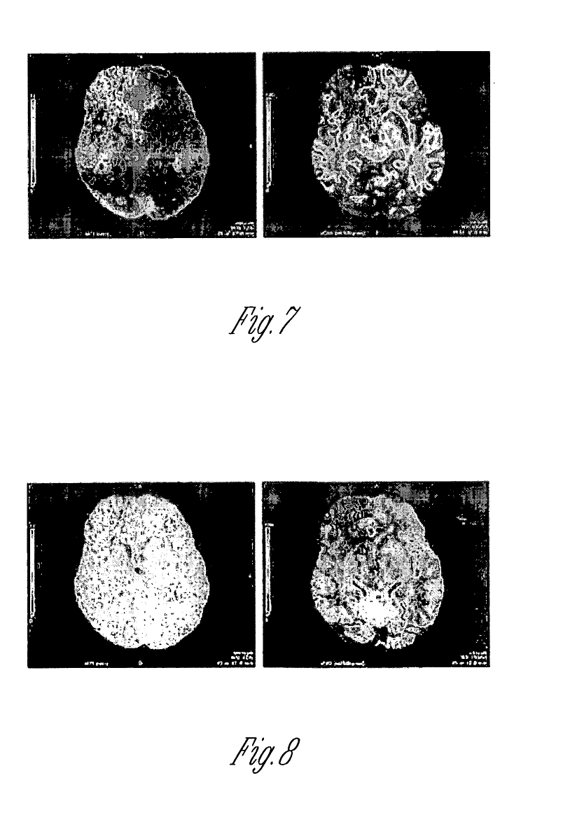

A real dataset was applied to the dSVDs technique with the standard SVD technique to illustrate the differences. The dataset contains 50 slices with a temporal resolution of 1 second. The artery location and the venous location were selected manually. The time of arrival of contrast to the artery curve is 6 seconds. Results determining cerebral blood flow (CBF) using the sSVD technique are shown in FIG. 7. The results for CBF obtained using the dSVDs technique are shown in FIG. 8.

In FIG. 7, a large area on the left side of the brain can be observed that has low MTT values and high CBF values. However, this area is not seen in FIG. 8 for the results computed using the dSVDs technique. The reason for this is that the selected artery is on the top right of the brain which has a time of arrival that is larger than those for the tissues on the left side of the brain, in other words, the artery curve itself is delayed, thus there is an underestimation of the MTT values using the sSVD technique as discussed previously. The dSVDs technique resolves this problem.

The delay insensitive SVD technique can be implemented in a medical imaging system. The dSVDs technique can be used for brain or other perfusion analysis, such as to determine indications of one or more of blood flow or mean transit time.

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAM's), read only memories (ROM's), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A computerized system comprising:
a memory circuit, configured to store a time sequence of volumetric image data, wherein the stored image data is obtained in a time relation to arrival of a contrast agent at an anatomical region associated with the image; and
a perfusion analyzer circuit, in communication with the memory circuit, wherein the perfusion analyzer is configured to:
access the stored image data and receive a representation of at least a portion of an artery included in the image data and a representation of tissue near the artery in the image data;
shift the representation of the artery in time such that a time of arrival of the contrast at the artery precedes arrival of the contrast at the representation of tissue;
automatically compute a perfusion parameter using the shifted representation of the artery; and
provide the perfusion parameter as an output to a user or to an automated process.

2. The system of claim 1, wherein the perfusion analyzer circuit comprises a mean transit time (MTT) computation circuit that is configured to determine a MTT of the contrast using the computed perfusion parameter.

3. The system of claim 2, wherein the MTT computation circuit comprises a filter circuit to inhibit oscillations in determined MTT values.

4. The system of claim 3, wherein the filter circuit is configured to extend a length of the representation of the artery by zero-padding to inhibit oscillations in determined MTT values.

5. The system of claim 1, wherein the perfusion analyzer circuit comprises a MTT computation circuit that is configured to:
precondition a system of linear equations to model a representation of infinite zero-padding of the shifted artery to inhibit oscillations in determined MTT values
solve the preconditioned system of linear equations to compute the perfusion parameter; and
determine values of MTT of the contrast using the perfusion parameter computed by solving the preconditioned system of linear equations.

6. The system of claim 5, wherein the representation of infinite zero-padding includes a Toeplitz matrix of values of the shifted artery.

7. The system of claim 5, wherein the perfusion analyzer circuit is configured to compute the perfusion parameter by solving a preconditioned system of linear equations using standard singular value decomposition.

8. The system of claim 1, wherein the perfusion analyzer circuit is configured to shift the representation of the artery such that the time of arrival of contrast at the artery curve is at a time equal to zero (t=0) for performing deconvolution.

9. The system of claim 1, wherein the perfusion analyzer circuit is configured to compute a contrast residue in a region of a stored image voxel as a function of time.

10. The system of claim 1, wherein the perfusion analyzer circuit is configured to automatically compute relative cerebral regional blood flow in a region of a stored image voxel using the perfusion parameter.

11. A computerized method comprising:
accessing a stored time sequence of volumetric image data, wherein the stored image data is obtained in a time relation to arrival of a contrast agent at a region of a subject;
receiving a representation of at least a portion of an artery in the stored image data and a representation of tissue near the artery in the image data;
shifting the representation of the artery in time such that a time of arrival of the contrast at the artery precedes arrival of the contrast at the representation of tissue;
automatically computing a perfusion parameter using the shifted representation of the artery; and
providing the perfusion parameter as an output to a user or to an automated process.

12. The method of claim 11, including:
determining a mean transit time (MTT) of the contrast using the computed perfusion parameter.

13. The method of claim 12, wherein shifting the representation of the artery includes adding zeros to an end of the representation of the artery to extend the artery to inhibit oscillations in determined MTT values.

14. The method of claim 12, including:
preconditioning a system of linear equations to include a representation to model infinite zero-padding of the shifted artery to inhibit oscillations in determined MTT values;
solving the preconditioned system of linear equations for computing the perfusion parameter; and
determining MTT of the contrast using the computed perfusion parameter.

15. The method of claim 14, wherein preconditioning the system of linear equations includes using a representation of infinite zero-padding that includes a Toeplitz matrix of values of the shifted artery.

16. The method of claim 14, wherein solving the preconditioned system of linear equations includes solving the system using standard singular value decomposition to compute the perfusion parameter.

17. The method of claim 11, wherein shifting the representation of the artery includes shifting such that the time of arrival of contrast at the artery curve is at a time equal to zero (t=0) for performing deconvolution.

18. The method of claim 11, wherein computing the perfusion parameter includes computing contrast residue in a region of a stored image voxel as a function of time.

19. The method of claim 11, including computing relative cerebral regional blood flow in a region of a stored image voxel using the perfusion parameter.

20. A computer-readable medium comprising instructions configured to perform a process comprising:
accessing a stored time sequence of image data, wherein the stored image data is obtained in a time relation to arrival of a contrast agent at a region of a subject;
receiving a representation of at least a portion of an artery in the stored image data;
shifting the representation of the artery curve in time such that a time of arrival of contrast at the artery precedes arrival of contrast at the representation of tissue;
automatically computing a perfusion parameter using the shifted representation of the artery; and
providing the perfusion parameter as an output to a user or to an automated process.

21. The computer-readable medium of claim 20, comprising instructions configured for computing relative cerebral regional blood flow in a region near a stored image voxel using the perfusion parameter.
